SEQUENCE LISTING (1 sequence)

SEQ ID NO: 1 - DNA, 1443 bp, Bacillus
ctggcggcgtgcctaatacatgcaagtcgagcggacagatgggagcttgctccctgatgttagcggcggacgggtgagtaacacgtgggtaacctgcctgtaagactgggataactccgggaaaccggggctaataccggatggttgtttgaaccgcatggttcagacataaaaggtggcttcggctaccacttacagatggacccgcggcgcattagctagttggtgaggtaacggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcaaatagggcggcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgataagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaacctttatggagccagccaccgaag

(19) United States
(12) Patent Application Publication
(10) Pub. No.: US 2008/0131953 A1
(43) Pub. Date: Jun. 5, 2008

(54) Bacterial Treatment

(76) Inventors: Terence Harden, New South Wales (AU); Steven Simpfendorfer, New South Wales (AU); Heather Cavanagh, New South Wales (AU); Leath Sheales, New South Wales (AU)

Correspondence Address: STINSON MORRISON HECKER LLP;ATTN: PATENT GROUP, 1201 WALNUT STREET, SUITE 2800, KANSAS CITY, MO, 64106-2150, US

(21) Appl. No.: 10/564,876

(22) PCT Filed: Jul. 16, 2004
(86) PCT No.: PCT/AU04/00962

§ 371 Date: Dec. 10, 2007

(30) Foreign Application Priority Data
Jul. 17, 2003 (AU) 2003904406
Feb. 19, 2004 (AU) 2004900839

(51) Int. Cl. C12N 1/20

(52) U.S. Cl. 435/252.5

(57) ABSTRACT

An isolated Bacillus strain for suppressing the growth of a fungus.

### FIELD OF THE INVENTION

The invention relates to use of bacteria for controlling growth of a fungus and to use of bacteria for treating a plant or animal having a fungal infection.

### BACKGROUND OF THE INVENTION

Many plants and animals are susceptible to infection by fungi. For example, pasture crops and subterranean clovers are susceptible to Phytophthora clandestina, Phythium irregulare, Fusarium acuminatum, Fusarium avenaceum, Collectrichium gleosporiodes, Sclerotinia minor and Scerotinia sclerotiorum; grain, pulse and oil crops are susceptible to Phytophthora medicaginis, Pythium ultimum, Fusarium culmorum, Fusarium graminearum, Cochliobulus saliva, Bipolaris sorokiniana, Gaeumannomyces graminis, Pleochaeta setosa and Rhizoctonia solani, fruit, vegetable and nut producing plants are susceptible to Phytophthora cactorum, Phytophthora citrophthora, Phytophthora cryptogea, Phytophthora erythroseptica, Phytophthora parasitica, Phytophthora sojae, Aphanomyces spp., Botrytis cinera, Collectrichium orbiculare, Monilinia fructicola, Monilia spp., Sclerotinia rolfsii, Aspergillus parasiticus, Aspergillus flavus, Lasiodiplodia theobromae, Phomopsis viticola, Mucor spp. and Aspergillus niger; and ornamentals and wood producing plants are susceptible to Phytophthora palmivora, Schizophyllum commune and Coriolus spp.

Many fungi infect animals and examples include those capable of infecting human skin: Microsporum gypseum, Microsporum canis, Trichophyton rubrum, Trichophyton mentagrophytes and Trichophyton tonsurans.

As few plants or animals are characterised by fungal resistance, the management and control of infection and disease often requires the use of chemical fungicides. A limitation applies to the use of fungicides because of the effect of such compounds on human health and the environment. Further, fungicides tend to be expensive.

There is a need for new products and processes for controlling growth of fungi that are capable of infecting plants and animals.

### SUMMARY OF THE INVENTION

The invention seeks to at least minimise one of the above limitations and in one aspect provides an isolated Bacillus strain for suppressing the growth of a fungus. The strain includes a nucleic acid molecule having the nucleotide sequence shown in SEQ ID NO:1 (FIG. 1), or a nucleic acid molecule that has at least 97% nucleotide sequence identity with a sequence shown in SEQ ID NO: 1.

In another aspect, the invention provides an isolated Bacillus stain for suppressing the growth of a fungus. The strain has a genome that has at least 70% DNA-DNA similarity to the genome of a Bacillus strain according to Accession no. NM03/36700 and a melting temperature that is about the same as the melting temperature of the genome of a Bacillus stain according to Accession no. NM03/36700.

In another aspect, the invention provides an isolated Bacillus strain according to Accession no. NM03/36700.

In another aspect, the invention provides a bacterial strain for suppressing the growth of a fungus. The stain is one produced by the following steps:
(a) exposing a strain as described above to conditions to produce one or more mutant strains; and
(b) selecting a mutant strain that is capable of suppressing the growth of a fungus and that has the nucleotide sequence shown in SEQ ID NO:1 (FIG. 1), or a nucleic acid molecule that has at least 97% nucleotide sequence identity with a sequence shown in SEQ ID NO:1.

In another aspect, the invention provides a bacterial strain for suppressing the growth of a fungus. The stain is one produced by the following steps:
(a) exposing a strain as described above to conditions to produce one or more mutant strains; and
(b) selecting a mutant stain that is capable of suppressing the growth of a fungus and that has a genome that has at least 70% DNA-DNA similarity to the genome of a Bacillus strain according to Accession no. NM03/36700 and a melting temperature that is about the same as the melting temperature of the genome of a Bacillus strain according to Accession no. NM03/36700.

In another aspect, the invention provides a composition for suppressing the growth of a fungus. The composition is characterised in being one produced by a Bacillus strain as described above.

In another aspect, the invention provides a process for producing a composition for suppressing the growth of a fungus. The process includes the step of maintaining a Bacillus strain as described above, in conditions for permitting the strain to produce the composition.

In another aspect, the invention provides a process for suppressing the growth of a fungus. The process includes the step of contacting the fungus with a Bacillus strain described above, or with a composition produced by the strain.

In another aspect, the invention provides a process for treating a fungal infection in a plant or animal. The process includes contacting the plant or animal with a Bacillus strain as described above, or with a composition produced by the strain.

### BRIEF DESCRIPTION OF THE DRAWINGS

FIG. 1. 16S rDNA sequence of C60.

FIG. 2. 16S rDNA extended-sequence phylogenetic tree.

FIG. 3. Configuration of bioassay.

### DETAILED DESCRIPTION OF THE EMBODIMENTS

As described herein, the inventor has found an isolate of bacteria according to Accession no. NM03/36700 that has utility in suppressing the growth of fungi that cause disease in plants and animals, including humans.

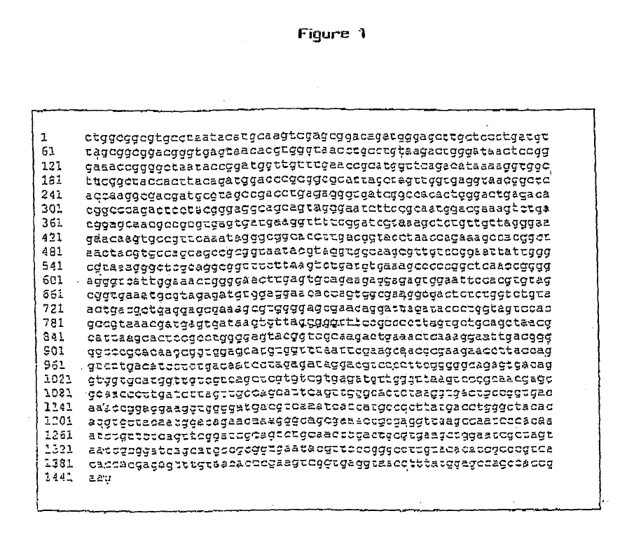

The inventor has identified the isolate as being within the genus Bacillus. Specifically, the inventor has found that the isolate contains a unique 16s ribosome DNA molecule that includes the sequence shown in FIG. 1. Although the sequence of the 16s ribosome DNA molecule is heretofore unknown, it is characteristic of the 16s ribosome DNA molecules of other Bacillus species. Accordingly, the inventor has found that the isolate is within the genus Bacillus.

The inventor has also found that the isolate is a new bacterial species. Specifically, by determining the relatedness of the nucleotide sequence of the genome of the isolate to the genomes of other known Bacillus species (i.e. determining DNA-DNA similarity of genomes), the inventor has found that the known Bacillus species have less than 70% similarity to the genome of the isolate. As described herein, the relatedness of a nucleotide sequence of an isolate to the genome of another species is determined by hybridising the genomes and measuring the amount of hybridisation between the genomes. As the known Bacillus species have less than 70% similarity to the genome of the isolate, the isolate is a heretofore unknown species of the genus, Bacillus.

The inventor believes that other strains of this species that are capable of suppressing the growth of fungus that cause disease in plants and animals could be identified by determining the sequence of the 16s ribosomal gene of these strains, and/or by determining the DNA-DNA similarity of the genomes of these strains to the genome of NM03/36700.

For example, a 16 ribosomal DNA molecule having a sequence shown in FIG. 1, or a molecule that has one or more points of nucleotide sequence difference to the sequence shown in FIG. 1, more particularly, at least 97% nucleotide sequence identity to the molecule shown in FIG. 1, could be used to identify the other strains of this species that are capable of suppressing the growth of fungus that cause disease in plants and animals.

Further, a genome that has at least 70% DNA-DNA similarity with a genome of a strain according to Accession no. NM03/36700 and a melting temperature that is about the same as the melting temperature of the genome of a Bacillus strain according to Accession no. NM03/36700 identifies other strains of the species that are capable of suppressing the growth of fungus that cause disease in plants and animals.

Thus it will be understood that a strain that is capable of suppressing the growth of fungus that causes disease in plants and animals, and that has a 16 ribosomal DNA molecule having a sequence that has at least 97% nucleotide sequence identity to the molecule shown in FIG. 1, or that has a genome that has at least 70% DNA-DNA similarity with a genome of a strain according to Accession no. NM03/36700 and a melting temperature that is about the same as the melting temperature of the genome of a Bacillus strain according to Accession no. NM03/36700, is a strain of the invention and is within the scope of the invention.

Accordingly, in one aspect, the invention provides an isolated Bacillus strain for suppressing the growth of a fungus, the strain including a nucleic acid molecule having the nucleotide sequence shown in FIG. 1.

In one embodiment, the strain includes a nucleic acid molecule that has at least 97% nucleotide sequence identity with the nucleotide sequence shown in FIG. 1. Preferably, the strain includes a nucleic acid molecule that has 98% sequence identity with the nucleotide sequence shown in FIG. 1. More preferably, the strain includes a nucleic acid molecule that has 99% sequence identity with the nucleotide sequence shown in FIG. 1.

In another aspect, the invention provides an isolated Bacillus strain for suppressing the growth of a fungus, the strain having a genome that has at least 70% DNA-DNA similarity to the genome of a Bacillus strain according to Accession no. NM03/36700 and a melting temperature that is about the same as the melting temperature of the genome of a Bacillus strain according; to Accession no. NM03/36700. Preferably the strain has a genome that has at least 75% DNA-DNA similarity to the genome of a Bacillus strain according to Accession no. NM03/36700. Preferably the strain has a genome that has at least 80% DNA-DNA similarity to the genome of a Bacillus strain according to Accession no. NM03/36700. Preferably the strain has a genome that has at least 85% DNA-DNA similarity to the genome of a Bacillus strain according to Accession no. NM03/36700. Preferably the strain has a genome that has at least 90% DNA-DNA similarity to the genome of a Bacillus strain according to Accession no. NM03/36700. Preferably the strain has a genome that has at least 95% DNA-DNA similarity to the genome of a Bacillus stain according to Accession no. NM03/36700.

In another aspect, the invention provides an isolated Bacillus strain according to Accession no. NM03/36700. The method for isolating this bacteria, its growth requirements and characteristics are described further herein. This bacteria has particular utility in inhibiting growth of fungi that cause disease in plants and animals, including humans.

One could isolate a strain of the invention, for example, one that includes a nucleic acid molecule having at least 97% nucleotide sequence identity with the nucleotide sequence shown in FIG. 1, or one having a genome that has at least 70% DNA-DNA similarity with a genome of a strain according to Accession no. NM03/36700 and a melting temperature that is about the same as that of NM03/36700, and that is capable of suppressing the growth of a fungus that causes disease in plants and animals, by screening such a strain for activity in the suppression of growth of fungi selected from the group consisting of Phytophthora clandestina, Phythium irregulare, Fusarium acuminatum, Fusarium avenaceum, Collectrichium gleosporiodes, Sclerotinia minor, Sclerotinia sclerotiorum, Phytophthora medicaginis, Pythium ultimum, Fusarium culmorum, Fusarium graminearum, Cochliobulus sativa, Bipolaris sorokiniana, Gaeumannomyces graminis, Pleochaeta setosa, Rhizoctonia solani, Phytophthora cactorum, Phytophthora citrophthora, Phytophthora cryptogea, Phytophthora erythroseptica, Phytophthora parasitica, Phytophthora sojae, Aphanomyces spp., Botrytis cinera, Collectrichium orbiculare, Monilinia fructicola, Monilia spp., Sclerotinia rolfsii, Aspergillus parasiticus, Aspergillus flavus, Lasiodiplodia theobromae, Phomopsis viticola, Mucor spp., Aspergillus niger, Phytophthora palmivora, Schizophyllum commune, Coriolis spp., Microsporum gypseum, Microsporum canis, Trichophyton rubrum and Trichophyton mentagrophytes. The sequence of the 16 s ribosomal gene and the % DNA-DNA similarity could be determined before or after the antifungal activity of the strain is determined.

In one embodiment, the strain of the invention is capable of suppressing growth of a fungus that is capable of growing on pasture crops and/or subterranean clovers. Examples of such fungi include Phytophthora clandestina, Phythium irregulare, Fusarium acuminatum, Fusarium avenaceum, Collectrichium gleosporiodes, Scerotinia minor and Sclerotinia sclerotiorum.

In another embodiment, the strain is capable of suppressing the growth of a fungus that is capable of growing on grain, pulse and/or oil crops. Examples of such fungi include Phytophthora medicaginis, Pythium ultimum, Fusarium culmorum, Fusarium graminearum, Cochliobulus sativa, Bipolaris sorokiniana, Gaeumannomyces graminis, Pleochaeta setosa and Rhizoctonia solani.

In another embodiment, the strain is capable of suppressing the growth of a fungus that is capable of growing on fruit, vegetable and/or nut producing plants. Examples of such fungi include Phytophthora cactorum, Phytophthora citrophthora, Phytophthora cryptogea, Phytophthora erythroseptica, Phytophthora parasitica, Phytophthora sojae, Aphanomyces spp., Botrytis cinera, Collectrichium orbiculare, Monilinia fructicola, Monilia spp., Sclerotinia rolfsii, Aspergillus parasiticus, Aspergillus flavus, Lasiodiplodia theobromae, Phomopsis viticola, Mucor spp. and Aspergillus niger.

In another embodiment, the strain is capable of suppressing the growth of a fungus that is capable of growing on ornamentals and/or wood producing plants. Examples of such fungi include Phytophthora palmivora, Schizophyllum commune and Coriolus spp.

In another embodiment, the strain is capable of suppressing the growth of a fungus that is capable of growing on human skin. Examples of such fungi include Microsporum gypseum, Microsporum canis, Trichophyton rubrum and Trichophyton mentagrophytes.

The inventor recognises that a strain that has a phenotype that is distinguished from the strain according to NM03/36700, or in other words, a strain having a mutant phenotype, and that has a capacity to suppress growth of fungi that cause disease in a plant or animal, such as those plants and animals described above, could be produced by following standard techniques. For example, one could produce a strain that has a capacity to suppress growth of fungi that cause disease in a plant or an animal and that has tolerance to UV, salt or heat, or that has antibiotic resistance, motility or endospore production. Processes for producing these strains are described further herein.

Thus in another aspect, the invention provides a bacterial strain for suppressing the growth of a fungus. The strain is one produced by the following steps:
(a) exposing a strain as described above to conditions to produce one or more mutant strains; and
(b) selecting a mutant strain that is capable of suppressing the growth of a fungus and that has the nucleotide sequence shown in FIG. 1, or a nucleic acid molecule that has at least 97% nucleotide sequence identity with a sequence shown in FIG. 1.

In yet another aspect, the invention provides a bacterial strain for suppressing the growth of a fungus. The strain is one produced by the following steps:
(a) exposing a strain as described above to conditions to produce one or more mutant strains; and
(b) selecting a mutant strain that is capable of suppressing the growth of a fungus and that has a genome that has at least 70% DNA-DNA similarity to the genome of a Bacillus strain according to Accession no. NM03/36700 and a melting temperature that is about the same as the melting temperature of the genome of a Bacillus strain according to Accession no. NM03/36700.

In one embodiment, the strain according to Accession no. NM03/36700 is exposed to conditions for producing a mutant strain that has salt tolerance.

In another embodiment, the strain according to Accession no. NM03/36700 is exposed to conditions for producing a mutant strain that has UV tolerance.

In another embodiment the stain according to Accession no. NM03/36700 is exposed to conditions for producing a mutant strain that has heat tolerance.

In another embodiment, the strain according to Accession no. NM03/36700 is exposed to conditions for producing a mutant strain that has antibiotic resistance.

It will be understood that the conditions for producing the mutant stain are those capable of producing the desired mutant phenotype. For example, the conditions for producing a mutant strain that has salt tolerance may include serial passage of the strain according to Accession no. NM03/36700 through media so as to expose the strain to increasing salt concentrations.

Alternatively, the conditions for producing the mutant strain may include introducing a nucleic acid molecule into the strain according to Accession no NM03/36700. This is a preferred approach for producing a strain that has antibiotic resistance.

Typically, the mutant strain is selected that is capable of suppressing the growth of a fungus selected from the group consisting of Phytophthora clandestina, Phythium irregulare, Fusarium acuminatum, Fusarium avenaceum, Collectrichium gleosporiodes, Sclerotinia minor, Sclerotinia sclerotiorum, Phytophthora medicaginis, Pythium ultimum, Fusarium culmorum, Fusarium graminearum, Cochliobulus sativa, Bipolaris sorokiniana; Gaeumannomyces graminis, Pleochaeta setosa, Rhizoctonia solani, Phytophthora cactorum, Phytophthora citrophthora, Phytophthora cryptogea, Phytophthora erythroseptica, Phytophthora parasitica, Phytophthora sojae, Aphanomyces spp., Botrytis cinera, Collectrichium orbiculare, Monilinia fructicola, Monilia spp., Sclerotinia rolfsii, Aspergillus parasiticus, Aspergillus flavus, Lasiodiplodia theobromae, Phomopsis viticola, Mucor spp., Aspergillus niger, Phytophthora palmivora, Schizophyllum commune, Coriolus spp., Microsporum gypseum, Microsporum canis, Trichophyton rubrum and Trichophyton mentagrophytes.

In another aspect, the invention provides a strain for suppressing growth of a fungus that causes disease in a plant or animal. The strain is characterised in being one derived from an isolate according to Accession no. NM03/36700. The strain may be derived by the steps of (a) and (b) described above.

In one embodiment the isolated strain is salt tolerant.

In another embodiment, the isolated strain is UV tolerant.

It will be understood that the strains of the invention may be provided in a composition in a granulated or powdered form. For example, they may be coated on a plant seed that is to be protected from fungal growth to provide a composition in powdered or granulated form.

They may also be provided in a composition as a liquid form.

These compositions may be adapted for dilution in a solvent such as water to provide a composition comprising strains of the invention at the desired concentration.

Methods for producing these compositions are described further herein.

As described herein, the inventor has analysed the suppression of fungal growth by strains of the invention and has found that at least one compound produced by these strains has an effect on growth suppression because the supernatant component from the culture of these bacteria has growth suppressive effects on fungi. The inventor has also shown that at least one compound produced by these bacteria is heat stable.

The inventor recognises that the compositions produced by the strains of the invention that have growth suppressive effects on fungi are particularly useful for suppressing fungal growth on plants and animals. In particular, the inventor recognises that these compositions will be particularly useful for suppressing growth of a fungus selected from the group consisting of Phytophthora clandestina, Phythium irregulare, Fusarium acuminatum, Fusarium avenaceum, Collectrichium gleosporiodes, Sclerotinia minor, Sclerotinia sclerotiorum, Phytophthora medicaginis, Pythium ultimum, Fusarium culmorum, Fusarium graminearum, Cochliobulus sativa, Bipolaris sorokiniana, Gaeumannomyces graminis, Pleochaeta setosa, Rhizoctonia solani, Phytophthora cactorum, Phytophthora citrophthora, Phytophthora cryptogea, Phytophthora erythroseptica, Phytophthora parasitica, Phytophthora sojae, Aphanomyces spp., Botrytis cinera, Collectrichium orbiculare, Monilinia fructicola, Monilia spp., Sclerotinia rolfsii, Aspergillus parasiticus, Aspergillus flavus, Lasiodiplodia theobromae, Phomopsis viticola, Mucor spp., Aspergillus niger, Phytophthora palmivora, Schizophyllum commune, Coriolus spp., Microsporum gypseum, Microsporum canis, Trichophyton rubrum and Trichophyton mentagrophytes, because the inventor has shown that the strains of the invention suppress the growth of these fungi.

Accordingly, in another aspect, the invention provides a process for producing a composition for suppressing the growth of a fungus at causes disease in a plant or animal including maintaining a strain of the invention in conditions for permitting the strain to produce the composition. These conditions are described further herein.

In another aspect, the invention provides a composition for suppressing growth of a fungus that is capable of growing on plants or animals, the composition being one produced by a strain of the invention. The strain of the invention may be according to Accession no. NM03/36700. The strain may be one including a nucleic acid molecule having the nucleotide sequence shown in FIG. 1, or a nucleic acid molecule that has at least 97% nucleotide sequence identity with a sequence shown in FIG. 1. The strain of the invention may be one including a genome that has at least 70% DNA-DNA similarity to the genome of a Bacillus strain according to Accession no. NM03/36700 and a melting temperature that is about the same as the melting temperature of the genome of a Bacillus strain according to Accession no. NM03/36700.

It will be understood that the compositions produced by the strains of the invention may be provided as a formulation in a granulated or powdered form. For example, they may be coated on a plant seed that is to be protected from fungal growth to provide a formulation in powdered or granulated form.

They may also be provided as a formulation in a liquid form.

These formulations may be adapted for dilution in a solvent such as water to provide the composition at the desired concentration.

Methods for producing these compositions are described further herein.

As described herein, the inventor has found that suppression of fungal growth, or in other words, inhibition, retarding or slowing or fungal growth, can be achieved by contacting a fungus with a strain of the invention, or a composition produced by the strain. For example, the inventor has found that a strain of the invention, or a composition produced by the strain is capable of suppressing the growth a fungus selected from the group consisting of Phytophthora clandestina, Phythium irregulare, Fusarium acuminatum, Fusarium avenaceum, Collectrichium gleosporiodes, Sclerotinia minor, Sclerotinia sclerotiorum, Phytophthora medicaginis, Pythium ultimum, Fusarium culmorum. Fusarium graminearum, Cochliobulus sativa, Bipolaris sorokiniana, Gaeumannomyces graminis, Pleochaeta setosa, Rhizoctonia solani, Phytophthora cactorum, Phytophthora citrophthora, Phytophthora cryptogea, Phytophthora erythroseptica, Phytophthora parasitica, Phytophthora sojae, Aphanomyces spp., Botrytis cinera, Collectrichium orbiculare, Monilinia fructicola, Monilia spp., Sclerotinia rolfsii, Aspergillus parasiticus, Aspergillus flavus, Lasiodiplodia theobromae, Phomopsis viticola, Mucor spp., Aspergillus niger, Phytophthora palmivora, Schizophyllum commune, Coriolus spp., Microsporum gypseum, Microsporum canis, Trichophyton rubrum and Trichophyton mentagrophytes.

Accordingly, in another aspect, the invention provides a process for suppressing the growth of a fungus that causes disease in a plant or animal, the process including the step of contacting the fungus with a stain of the invention or a composition produced by the strain.

In another aspect, the invention provides a process for treating a fungal infection in a plant or animal, the process including contacting the plant or animal with a strain of the invention, or with a composition produced by the strain.

In the above described processes, the strain of the invention may be according to Accession no. NM03/36700. The strain may be one including a nucleic acid molecule having the nucleotide sequence shown in FIG. 1, or a nucleic acid molecule that has at least 97% nucleotide sequence identity with a sequence shown in FIG. 1. The strain of the invention may be one including a genome that has at least 70% DNA-DNA similarity to the genome of a Bacillus strain according to Accession no. NM03/36700 and a melting temperature that is about the same as the melting temperature of the genome of a Bacillus strain according to Accession no. NM03/36700.

In agricultural applications, a strain of the invention can be applied by sprays, seed coats, granular applications etc.

In medical or veterinary applications, a composition produced by a strain of the invention can be applied topically, for example as a cream, lotion, spray or inhalant.

The foregoing describes embodiments of the present invention and modifications, obvious to those skilled in the art can be made thereto, without departing from the scope of the present invention.

### Example 1

### Identification of C60

Partial 168 rDNA and extended 16S rDNA sequences were obtained via commercial testing at the Centre for Bacterial Diversity and Identification, Australian Collection of Microorganisms, School of Molecular and Microbial Sciences, The University of Queensland, Australia.

A culture of C60 stain was grown in peptone yeast extract broth (PYE) at 37° C. for 16 hours. Genomic DNA was extracted from a 5 mL broth culture using a FastDNA Spin Kit for Soil (BIO101) according to the manufacturer's instructions. The extracted DNA was separated using a 1% agarose (1×TAE) gel. 16S PCR was performed using the 27F and 1429R primers, the sequences of which are shown in Table 1.

TABLE 1

Primer sequences for 16S rDNA bacterial
identification

| | |
|---|---|
| Primer | Sequence |

| | |
|---|---|
| 27F | AGA GTT TGA TCM TGG CTC AG |
| 1429R | TAC GGY TAC CTT GTT ACG GAC TT |
| 907R | CCG TCA ATT CMT TTR AGT TT |

M = C or A; Y = C or T; R = A or G

PCR amplification was performed initially in volumes of 25 μL containing a final concentration of 1× reaction buffer (Promega), IA 14M MgCl₂, 200 μM dNTPs, 200 ng/μL 27F and 1492R (Invitrogen), 1 U AmpiTaq Gold polymerase (Promega) and 2 μL DNA template. Amplification was performed using a DNA Thermal Cycler (Hybaid OmniGene, Integrated Sciences). The cycling conditions for the PCR are described in Table 2.

TABLE 2
PCR thermal cycling conditions for 16S amplification

| Step | | |
|---|---|---|
| Number | Temperature (° C.) | Time (minutes) |
| 1 | 96 | 9 |
| 2 | 48 | 1 |
| 3 | 72 | 2 |
| 4 | 96 | 1 |
| 5 | Go to Step 2 for 30 cycles | |
| 6 | 48 | 1 |
| 7 | 72 | 5 |
| 8 | 25 | Hold |

The products of the PCR were separated by gel electrophoresis on a 1% agarose (Progen Cat #200-0011) gels prepared using 1× Tris-acetate EDTA (TAE) buffer (40 mM Tris-acetate, 2.5 mM EDTA—pH 8.0), containing 1 μg/mL ethidium bromide. The gels were visualised using UV illumination. Positive controls were run for all PCR procedures while the negative control consisted of the reaction mixture with no added template.

The presence of a 1,500 bp band for the sample and the positive control was confirmed by comparison with a molecular weight marker (100 bp ladder: Promega). The 16S procedure was then repeated using 100 μL reaction mixture volumes for increased product for purification. Following this amplification procedure the 16S product was purified using a Wizard Purification Kit following the manufacturer's protocol and a sequencing reaction performed.

The sequencing reaction was performed using a protocol from ABI. Briefly, the amplification was performed in 10 μL volume reactions, consisting of 4 μL Big Dye Terminator reaction mix (BDT Version 2.0), 0.5 μL 200 ng/μL primer, 1.5 μL purified 16S product and 4 μL sterile water. Separate reactions were performed using three primers, 27F, 907R and 1429R, as listed in Table 1. A DNA Thermal Cycler was then used to amplify the DNA following cycling conditions as listed in Table 3.

TABLE 3
PCR cycling conditions for sequencing amplification

| Step | | |
|---|---|---|
| Number | Temperature (° C.) | Time |
| 1 | 96 | 2 minutes |
| 2 | 96 | 30 seconds |
| 3 | 50 | 15 seconds |
| 4 | 60 | 4 minutes |
| 5 | Go to Step 2 for 24 cycles | |
| 6 | 25 | Hold |

After amplification the extension products of the sequencing reaction were purified using the isopropanol protocol recommended by the ABI. Purified final products were submitted to the Australian Genome Research Facility for gel separation.

The results of a DNA analysis are shown in FIG. 1.

A partial sequencing yielded a 700 base pair (bp) sequence. Analysis of the partial DNA sequence gave a range of organisms that could be related to the unknown bacterial strain however the C60 strain did not have a complete identity with any recorded species. Extended DNA sequence analysis was carried out to further identify the closest phylogenetic relatives of the unknown strain. The extended DNA sequence obtained was 1,440 bp (FIG. 1).

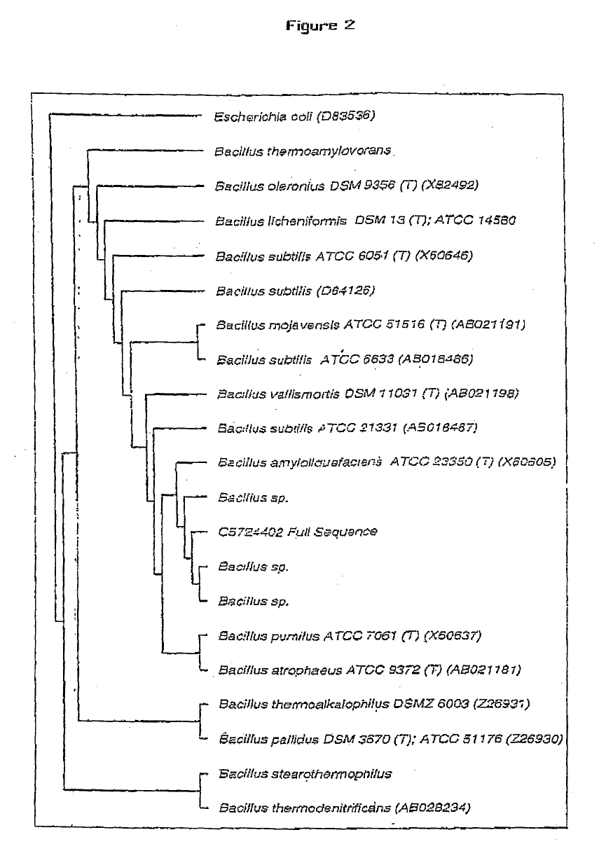

From the extended DNA sequence the bacterial strain can be positioned into a phylogenetic tree according to the relative homology with the DNA sequences of other bacterial species (FIG. 2). C60 is listed as ‘C5724402 Full Sequence’ in this figure.

The identified, named bacterial species showing the closest genetic similarity to C60 are Bacillus subtilis (ATCC 21331) and Bacillus amyloliquefaciens (ATCC 23350).

In addition to the phylogenetic tree, a similarity matrix was generated to indicate the percentage identity between the C60 strain and those known sequences. This similarity matrix (Table 4) indicates the percentage DNA matches of 99.72% for B. subtilis 81 and 99.71% for B. amyloliquefaciens. Bacillus spp. 151, 152 and 154 all had 99.86% similarity to species C60, but neither has been localised to belonging to a given species of the Bacillus genus.

TABLE 4
Genetic similarity of 16 S of C60 to other bacterial strains

| Bacterial Strain | C60 % Genetic Match |
|---|---|
| C60 | 100.00 |
| Bacillus sp. 151 | 99.86 |
| Bacillus sp. 154 | 99.86 |
| Bacillus sp. 152 | 99.86 |
| B. subtilis 81 | 99.72 |
| B. amyloliquefaciens | 99.71 |
| B. vallismortis | 99.65 |
| B. subtilis 65 | 99.56 |
| B. atrophaeus | 99.43 |
| B. mojavensis | 99.43 |
| B. subtilis 79 | 99.36 |
| B. subtilis 22 | 99.29 |
| B. licheniformis | 98.23 |
| B. pumilus | 97.45 |
| B. oleronius | 95.18 |
| B. pallidus | 93.41 |
| B. thermoalkalophilus | 93.30 |
| B. denitrificans | 92.35 |
| B. stearothermophilus | 92.19 |
| B. thermoamylovorans | 91.91 |
| E. coli | 78.00 |

In summary, FIG. 2 and Table 4 both suggest that C60 is a member of the genus, Bacillus.

We next sought to determine whether C60 is a member of a known Bacillus species by performing a DNA-DNA hybridisation assay of the C60 genome against the genomes of the following bacterial species: Bacillus amyloliquefaciens Deutsche Sammlung von Mikroorganismen und Zelkulruren GmbH, Germany (herein “DSM”) 7, Bacillus subtilis subsp. spizizenii DSM 347, Bacillus subtilis subsp. subtilis DSM 10; Bacillus licheniformis DSM 13; Bacillus vallismortis DSM 11031, Bacillus atrophaeus DSM 675, Bacillus pumilus DSM 27 and Bacillus mojavensis DSM 9205.

The DNA-DNA analysis was performed by isolating DNA from the above described bacterial strains using a French pressure cell and was purified by chromatography on hydroxyapatite as described by Cashion et al. 1977 Anal. Biochem. 81: 461-466. DNA-DNA hybridization was carried out as described by De Ley et al. 1970 Eur. J. Biochem. 12: 133-142, with the modifications described by Huss et al. 1983 System. Appl. Microbiol. 4: 184-192 and Escara & Hutton 1980 Biopolymers 19: 1315-1327, using a model 2600 spectrophotometer equipped with a model 2527-R thermoprogrammer and plotter (Gilford Instrument Laboratories). Renaturation rates were computed with the TRANSFER.BAS program of Jahnke and Bahnweg 1986 Trans. Br. My. Soc. 87: 175-191.

In the results shown in Table 4A, Bacillus sp. showed a moderate DNA-DNA similarity (67.6, rep. 62.6%) to Bacillus amyloliquefaciens DSM7, which is just below the threshold value of 70% for definition of bacterial species. None of the other DNA-DNA hybridisations indicated a relationship at the species level. In summary, the DNA-DNA hybridisation analysis suggests that C60 represents a heretofore unknown Bacillus species.

TABLE 4A
% DNA-DNA similarity (in 2xSSC at 65° C.)

| | % DNA-DNA |
|---|---|
| Bacterial genome | similarity to C60 |
| Bacillus amyloliquefaciens DSM 7 | 67.6; 62.6 |
| Bacillus subtilis subsp. spizizenii DSM 347 | 27.6 |
| Bacillus subtilis subsp. subtilis DSM 10 | 22.8 |
| Bacillus licheniformis DSM 13 | 24.5 |
| Bacillus vallismortis DSM 11031 | 29.5 |
| Bacillus atrophaeus DSM 675 | 18.4 |
| Bacillus pumilus DSM 27 | 28.3 |
| Bacillus mojavensis DSM 9205. | 27.1 |

The presence or absence of plasmids in C60 was determined using a TempliPhi 100 Amplification Kit (Amersham Biosciences 25-6400-10). Bacterial cultures of C60 was grown in 10 mL NB overnight before being centrifuged at 10,000 rpm in a Beckman J2-21M/E centrifuge for 4 minutes. The supernatant was discarded and the bacterial pellets resuspended in the residual volume, henceforth referred to as saturated bacterial cultures. 1 μL saturated bacterial culture was added to 5 μL sample buffer, after which the tube was capped and heated to 95° C. for 3 minutes then cooled to room temperature. 5 μL reaction buffer was mixed with 0.2 μL enzyme mix and added to the microcentrifuge tube. The tube was vortexed briefly and incubated at 30° C. for 445 hours. The sample was then heated to 65° C. for 10 minutes and cooled to 4° C. to inactivate the enzymes. Positive controls were prepared using positive control sample provided by the manufacturer in place of bacterial cultures.

Amplified sample was digested using the restriction enzymes BamH1 and Hind III. 10 units of each enzyme (1 μL) was added to 5 μL sample plus 13 μL buffer in a sterile eppendorf tube. Digest mixtures were incubated at 37° C. for one hour before being removed for electrophoretic visualisation of a plasmid band.

The electrophoresis gel was prepared by adding 1.5% w/v electrophoresis grade agarose (Progen Cat. #200-0011) in TB buffer. The mixture was heated to boiling in a microwave oven to dissolve the agarose before being removed and cooled to 60° C. 10 μL ethidium bromide stock solution (20 μg/mL) was added to the cooled agarose solution and poured into a large gel tray with a 20 well comb inserted. The gel was allowed to set before being placed into an electrophoresis tank (Bio-Rad) and filled with sufficient TB buffer to cover the gel (approximately 2 L). Restriction enzyme digested samples were mixed with 2 μL 6× loading dye (Promega G188A). Lambda bacteriophage DNA marker digested with Hind III (λHindIII) was used to estimate the size of any bands on the gel. A constant 100 V was applied across the gel and the separation was run for 1 hour, after which the gel was removed from the electrophoresis tank and visualised by fluorescence under UV light and photographed.

In this analysis, the positive controls gave a single band, indicating that plasmid DNA was successfully amplified. No evidence of extrachromosomal plasmids were detected in C60. The method utilised amplifies plasmids regardless of size or copy number, therefore the absence of amplified bands, in the presence of positive controls, indicates that C60 is unlikely to contain plasmids.

### Example 2

### Suppression of Fungal Growth Using C60 or a Composition Obtained Therefrom

A suspension of Bacillus C60 was prepared using one of two methods, both of which were compared for consistency. The first method used C60 prepared from 24-48 hour cultures grown on nutrient agar (NA) at 25° C. using a streak plate method. The C60 bacteria were harvested from the plate by washing with 5 mL of sterile phosphate buffered saline (PBS) and gentle scraping of the surface of the agar with a sterile glass spreader. The resulting suspensions were placed into sterile 10 mL plastic tubes, vortexed for two minutes to disperse clumps and adjusted to an absorbance of 0.6 at 625 nm with sterile PBS. This gave an approximate concentration of 1×10⁶ cells/mL, verified by pour plate. The cell suspension was diluted 10-fold to a dilution of 1×10⁻⁷. 1 mL of each dilution was poured into a sterile petri dish and overlayed with 20 mL sterile NA at 45° C. The plates were swirled to disperse the C60 bacteria and allowed to set before being incubated at 25° C. for 24 hours. The plates were examined and the number of colonies counted, confirming the relationship between 0.6 absorbance units at 625 nm and 1×10⁶ cells/mL.

The second method of preparing C60 suspensions began by inoculating 15 mL sterile nutrient broth (NB) with a loop-full of C60. Broth cultures were incubated at 25° C. for 24-48 hours and were harvested by centrifugation at 4,000 rpm for 10 minutes. The supernatant was discarded and the C60 cells resuspended in 5 mL sterile PBS. The absorbance of the suspension was adjusted to 0.6 at 625 nm with sterile PBS, giving an approximate concentration of 1×10⁶ cells/mL.

Frozen aliquots of stored C60 cultures were prepared by inoculating 100 mL sterile nutrient broth (NB) with a fresh inoculating loop-full of C60 colonies and incubated at 25° C. with shaking for 24 hours. This starter culture was then added to 900 mL sterile NB and further incubated at 25° C. for 48 hours. Following incubation, 10 mL aliquots of the C60 suspension were aseptically transferred to sterile tubes and stored frozen at −20° C. These aliquots were removed from storage, thawed and added to 90 mL sterile NB and incubated at 25° C. for 24-48 hours to produce additional C60 starter cultures as required. C60 bacteria were also stored on NA slopes at 4° C., and cultured fresh onto NA plates using streaking technique prior to being harvested by glass spreader or inoculating into 15 mL NB.

Both harvesting methods were used in comparison of inhibition of the growth of fungal pathogens. No difference was observed in the inhibition of pathogens between the C60 bacteria derived from different harvesting methods.

Fungal pathogens were prepared by inoculating NA, potato dextrose agar (PDA), Sabaroud's agar (SAB) or V8 Juice+0.2% CaCO₃ agar (V8) (20 Ml agar per 90 mm diameter petri dish). Agar plates were inoculated with a plug of fungus cut from the growing edge of a colony of the fungus using a sterile cork borer (7 mm diameter). The agar used for each pathogen is listed in Table 5. The plates were edge wrapped with Parafilm and incubated at 25° C. After a number of days, as indicated in Table 5, plugs of fungus were cut from the growing colony and used to inoculate bioassay plates, or transferred to a sterile agar plate for preparation of additional fungal inoculum.

TABLE 5
Days of growth before harvesting fungal plugs for use in bioassay

| Fungus | Growth Period (days) | Agar Used |
|---|---|---|
| Aspergillus niger | 2 | NA |
| Bipolaris sorokiniana | 3 | PDA |
| Epidermophyton flocossum | 5 | SAB |
| Fusarium culmorum | 2 | PDA |
| Fusarium graminearum | 1 | PDA |
| Leptosphaeria maculans | 3 | V8 |
| Microsporum canis | 3 | SAB |
| Microsporum gypseum | 3 | SAB |
| Phomopsis viticola | 5 | PDA |
| Pleochaeta setosa | 3 | PDA |
| Sclerotinia sclerotiorum | 3 | PDA |
| Trichophyton mentagrophytes | 3 | SAB |
| Trichophyton rubrum | 5 | SAB |
| Trichophyton tonsurans | 5 | SAB |

Fungal cultures were stored at 4° C. on agar plates edge-wrapped with Parafilm (agar type specified in Table 5). Fresh fungal colonies were subcultured every two weeks from the stored plates to maintain viability.

The in vitro inhibition of pathogenic fungi was tested using a modified paired in vitro diffusion bioassay. Briefly, inhibition of pathogenic fungi in vitro was assessed on 20 mL NA, PDA, SAB, or V8 agar plates.

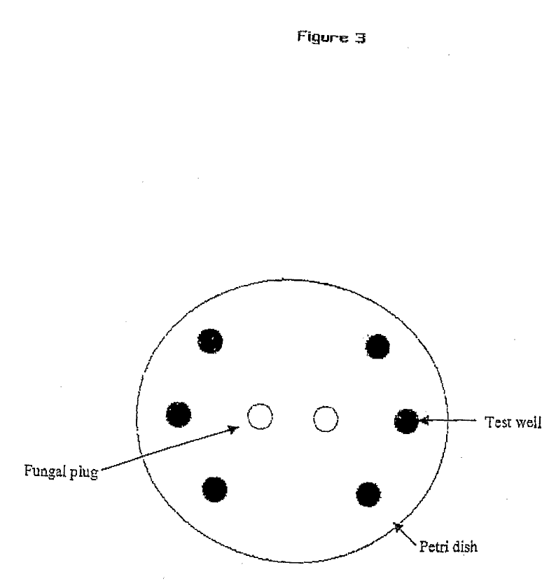

Once the agar had solidified, plugs were cut from the edges of each agar plate using a 7 mm cork borer as illustrated in FIG. 3 and the resulting wells inoculated with 100 μL of the C60 bacterial suspension. Control wells contained 100 μL sterile PBS in place of the test bacterial suspension.

After the addition of the C60 bacterial suspension or PBS to the wells, the plates were left at room temperature for 16 hours to allow the liquid to absorb into the agar. Two mycelil plugs of 7 mm diameter were cut from the margin of actively growing fungal colonies and inoculated onto the bioassay plate at equal distances (20 mm) from each grouping of wells, as indicated in FIG. 3. The plates were sealed and incubated in darkness at 25° C., with the distance between edge of colony growth and the well measured daily. Three independent replicates of each bacterial isolate and fungus combination and control fungal plates were prepared.

Due to differences in growth rates of different fungal pathogens, fungal growth data was converted to relative percentage inhibition of growth as compared to the growth of fungal colonies on control plates. Comparison of the sample means of fungal colony growth to growth of colonies on control plates was performed using an unpaired Student's t-test.

The antifungal metabolites produced by C60 bacteria are able to inhibit a broad range of fungal pathogens, corresponding to several different families of fungi and host types as shown in Table 6.

TABLE 6
Percentage inhibition of fungal pathogens by
metabolites produced by live C60 bacteria

| Fungal Pathogen | % Inhibition due to C60 Bacterial |
|---|---|
| A. niger | 89 ± 2% |
| B. sorokiniana | 66 ± 4% |
| E. floccosum | 96 ± 5% |
| F. culmorum | — |
| F. graminearum | 32 ± 13% |
| L. moculans | 100 ± 0% |
| M. canis | 93 ± 7% |
| M. gypseum | 91 ± 12% |
| P. viticola | 35 ± 6% |
| S. sclerotiorum | 63 ± 6% |
| T. mentagrophytes | 92 ± 5% |
| T. rubrum | 89 ± 9% |
| T. tonsurans | 90 ± 11% |

The C60 bacteria exhibited a maximum inhibitory activity against L. maculans (100±0%) and a minimum activity against F. graminearum (32±13%).

C60 bacteria were grown on nutrient agar which was then centrifuged to product a cell-free supernatant which was tested for antifungal activity. The C60 bacterial isolate was heavily streaked onto 2 separate nutrient agar plates and grown at 25° C. for 4 days.

The agar containing the C60 bacterial growth was the homogenised and centrifuged at 17,000 rpm for 15 minutes at 10° C. The resulting C60 supernatant was then passed trough a 0.22 μm filter to remove any C60 bacterial cells. The antifungal agent was held at temperatures ranging from 40° C. to 100° C. for 15 minutes.

Three 7 mm diameter wells were cut into each end of PDA plates. 100 μL of heated C60 supernatant solution was pipetted into each well and allowed to diffuse into the agar until the well appeared dry (overnight). Control plates consisted of homogenised and filtered nutrient agar that was not inoculated with any C60 bacteria.

Two mycelial disks (7 mm diameter) of Gaeumannomyces graminis were then inoculated 20 mm away from the antifungal wells and incubated at 20° C.

Inhibition was measured as mycelial growth directly toward the antifungal wells and maximum mycelial growth away from the antifungal wells. All figures are an average of three replicate plates.

TABLE 7

C60

| Temperature | Max | Min |
|---|---|---|
| 40° C. | 80% | 70% |
| 50° C. | 80% | 61% |
| 60° C. | 90% | 93% |
| 70° C. | 90% | 77% |
| 80° C. | 88% | 10% |
| 90° C. | 88% | 50% |
| 100° C. | 90% | 74% |

Figures represent the % inhibition in mycelial growth of Gaeumannomyces graminis. These results demonstrate that the antifungal agent produced by C60 is heat stable, indicating that it is not a protein.

The inhibition of the organisms listed in Table 8 by C60 cell-free filtrates was also examined. It was found that cell-free filtrates from C60 bacteria inhibited X. campestris. A zone of inhibition was considered to exist if the organism was unable to grow closer than 1 mm from the streak of test bacteria. The results of inhibition due to live bacterial cells are detailed in Table 8.

TABLE 8
Inhibition of bacteria and yeast by the presence of C60 bacteria

| | Presence or Absence of |
|---|---|
| Microorganism | Growth due to C60 |
| Alcaligens faecalis | − |
| Bacillus stearothermophilus | − |
| Bacillus subtilis | − |
| Escherichia coli | − |
| Mycobacterium phleti | − |
| Proteus mirabilis | − |
| Pseudomonax aeruginosa | − |
| Saccharomyces cerevisiae | − |
| Salmonella enteritidis | − |
| Serratia marcescens | − |
| Shigella sonnei | − |
| Xanthomonas campestris | + |

Only X. campestris was inhibited by C60. As a zone of inhibition was evident in the growth of X. campestris due to both colonies of the C60 bacteria as well as around cell-free culture filtrate, C60 is likely to be producing compounds inhibitory to the growth of this bacteria. The single yeast species tested was not inhibited by either the presence of growing C60 bacterial cultures nor the C60 cell-free filtrate.

### Example 3

### Screening of Biological Control Potential

Screening was performed against a Race 1 P. clandestina Isolate.

P. clandestine was present in the pathogen pots at a 2.5% w/w (inoculum:peat moss/sand) concentration. The fugal inoculum was grown on fine-grade vermiculite (sieved through pore size 2 mm) amended with Black-eye Bean+0.2% CaCo₃ Broth (2 L of broth per 1 kg of vermiculite) in 250 mL conical flasks for 3 weeks at 25° C. The colonised vermiculite was uniformly incorporated into a steam pasteurised (60° C. for 1 hr) potting mix (1:1 peat moss to river sand) at a rate of 2.5% (w/w). Uninfested vermiculite soaked in black-eye bean broth was added to the potting mix as a control ie, niI P. clandestina treatment.

C60 was applied as a bacterial suspension soaked onto the roots of the P. clandestina susceptible cultivar Woogenellup. C60 bacterial suspension was prepared from two 24-48 hr cultures streaked onto nutrient agar plates. The C60 bacteria were washed off each plate with 5 mL of sterile phosphate buffered saline and the resulting 10 mL C60 bacterial suspension placed into a sterile 1 oz. McCartney bottle. 60 germinated seeds (radicle˜2 mm) of cv. Woogenellup were then soaked overnight in C60 bacterial suspension (˜10⁶ cells/mL) before sowing.

All screening was performed in 10 cm black plastic pots with 3 replicate pots per treatment with a total of 10 non-bacterial or C60 bacterial treated seeds being sown per pot.

Treatments:

i. C60 bacteria only

ii. C60 bacteria plus P. clandestine

iii. Nil P. clandestina control

iv. P. clandestina only control.

24 hrs after sowing, all pots were watered by flooding with tap water for 2 hrs by filling an outer container which holds each pot. The pots were then flooded at weekly intervals. Disease suppression was assessed after 20 days growth in the glasshouse (temperature maintained between 15 to 25° C.) using the following measurements:

$$i ) Survival \text{:} \frac{number of plants present in each pot}{number of seeds sown in each pot ( 10 )} \times 100$$

ii) Average fresh plant weight: Measured by washing the potting mix away from the roots of surviving seedlings and blotting dry with paper towelling.

$$Average fresh plant weight ( per pot ) = \frac{Total mass ( mg ) of plants}{number of surviving plants}$$

iii) Average dry plant weight: Seedlings were oven dried in separate paper bags for the contents of each pot at 70° C. for 2 days and then weighed. Average dry plant weight then calculated as per fresh plant weight.

$$Average fresh plant weight ( per pot ) = \frac{Total dry mass ( mg ) of plants}{number of surviving plants}$$

iv) Root disease index: Twenty days after sowing, which has been reported as sufficient time for P. clandestina to produce disease, the surviving seedlings were counted and rated for root rot severity on the tap roots, using a 0-5 scale:

0, tap root healthy;

0.5, parts of tap root light brown;

1.0, whole root light brown;

1.5, part of root brown;

2.0, whole root brown;

2.5, root brown with visible lesions;

3.0, as for 2.5 with tip of the root constricted;

3.5, root brown and stunted;

4.0, root dark brown, discrete lesions may be present;

4.5, root dark brown, tissues beginning to slough off;

5.0, whole root rotted off or seedling dead.

The average percentage root disease index was then calculated using the method,

$$\frac{sum of all numerical ratings \times 100}{total number of inoculated plants \times 5}$$

v) Additionally randomly selected root pieces from each treatment were surface-sterilised in 70% ethanol for 2-3 minutes and rinsed three times in sterile deionised water. The root sections were then floated in sterile deionised water to induce the formation of P. clandestina oospores and sporangia. The presence of these structures in rot-affected tissue was therefore concluded to be evidence of P. clandestina being the primary cause of the observed root rot.

TABLE 9
Summary of Glasshouse screening results
against PC15 (Race 1 P. clandestina)

| | Survival | | Fresh Plant | | Dry Plant | |
|---|---|---|---|---|---|---|
| Treatment | (%) | | Weight (mg) | | Weight (mg) | |
| Nil pc 15 | 100 | (−) | 308.6 | (−) | 26.0 | (−) |
| Control | | | | | | |
| pc 15 Only | 73 | (−) | 76.1 | (−) | 7.2 | (−) |
| Control | | | | | | |
| C60 | 93 | (−7%) | 238.2 | (−23%) | 17.4 | (−33%) |
| C60 + Pc15 | 77 | (+5%) | 137.9 | (+81%) | 12.2 | (+69%) |

Figures in brackets represent percentage increase or decrease compared with the controls.

Referring to Table 9, it is evident that C60 bacteria significantly limit the severity of P. clandestina induced root rot of subterranean clover.

### Example 4

### Coating Seeds with C60 Bacteria

C60 bacteria was coated as a concentrated bacterial suspension onto sterilised seeds of cv. Woogenellup. Concentrated C60 bacterial suspension was prepared from two 72 hr cultures of C60 bacteria streaked onto nutrient agar plates. The C60 bacteria were washed off each plate with 5 mL of sterile phosphate buffered saline and the resulting 10 mL C60 bacterial suspension centrifuged 5,000 rpm for 5 minutes to pellet the C60 bacteria. 60 surface sterilised seeds (70% ethanol 10 minutes, followed by 5 washes with sterile deionised water) were placed into each bacterial pellet along with 2 mL of sterile 1% methylcellulose and some talcum powder. Tubes were then vigorously vortexed for 2 minutes to evenly coat seed. Coated seeds were then dried under a sterile continuous air flow (laminar flow hood) and broken up into individual coated seeds. C60 bacterial counts were around 10⁶ cells per seed. (NB: Talcum powder prevents the seeds becoming one mass of seeds, that is, it aids in the separation of seeds). Control seeds consisted of 2 mL of sterile deionised water instead of a bacterial pellet. Seeds were then coated with methylcellulose and talcum powder as per seeds treated with bacterium.

### Example 5

### C60 Bacteria Liquid Formulations

Bacteria were grown in 2×1 L nutrient broth shake cultures at 25° C. in a shaking orbital air incubator (5 rpm shaking speed) for 96 hours.

Bacteria were then pelleted by centrifugation at 7,500 rpm at 10° C. The resulting bacterial pellet was then reconstituted up to 10 mL with sterile phosphate buffered saline and vigorously vortexed for 2 minutes to breakup the pellet.

5 mL of the concentrated bacterial suspension was evenly distributed (1.6 mL per bottle) into 3 replicate 400 mL 0.1M MgSO₄ solutions. These bacterial suspensions then served as the solutions for spray inoculation of the biological control agents. The suspensions contained ˜10⁶ cells per mL.

### Example 6

### Field Trials

Two sites: one irrigation and one dryland are selected for field trials. Both sites have been previously determined by soil baiting to contain moderately high levels of P. clandestina naturally present in the soil.

3 replicate, 1 metre by ½ metre hand sown plots were established for each treatment. Ten (50 cm long) rows (furrows) were scratched into each plot with 50 seeds being sown into each row (ie, total of 500 seeds per plot). The field trial was divided into three large rows with a 1 metre gap between each row. One replicate of each treatment was randomly sown within each of these three rows. Plots within a row were separated from each other by a ½ metre gap.

Bacterial biological control agents were prepared for the field trial according to Example 4 and Example 5.

Seeds to be sown into each plot were dispensed into 10 separate sterile 10 mL tubes. The 50 seeds to be placed into each tube was measured using an average weight measurement for subclover seeds ie, 50 uncoated seeds=0.5 g, whilst 50 seed coated seeds=0.6-0.7 g. Therefore, when sowing the mini-plots for the field trial it was simply a matter of evenly sprinkling the contents (50 seeds) of an individual tube into each of the 10 furrows within a plot.

Uncoated seeds were sown into each furrow within a plot and evenly sprayed with 400 mL of bacterial solution (bacteria in 0.1M MgSO₄) before covering over with soil. The seeds coated with bacteria were sprinkled straight into the furrows before covering with soil.

Fungicide control treatment of seeds was included to assess the extent of root rot resident in the field site. The fungicide control consisted of sowing the seeds into each furrow within the plot before sprinkling 5 g of Ridomil (active ingredient is metalaxyl) evenly over the 10 furrows and covering with soil.

Disease suppression by the three bacteria and the fungicide treatment was measured at 4 and 8 weeks after sowing in terms of survival, root disease index, fresh and dry plant weights. The long term effects of bacteria on plant growth was assessed at 8 months using a rising plate count.

Three rising plate counts were randomly taken within each plot. The average rising plate count for the three replicate plots for each treatment could then be converted to a yield measurement using a standard curve of rising plate height versus dry weight (mg).

Data is expressed as a % increase in dry plant weight over nil bacterial controls.

### Example 7

### Producing Mutant Strains of C60 Bacteria

Salt Tolerance.

Most bacteria prefer to grow in conditions of between 0.5%-3% salt. Salt concentrations of 10%-15% are inhibitory to the growth of most bacteria, excluding halophiles. By growing C60 on agar with sufficient nutrients and a salt concentration of around 3%, it is possible to select for those members of C60 that are tolerant of salt conditions in the upper limits. By selecting those colonies and inoculating them onto nutrient agar with higher salt concentration (say, 4%) those C60 bacteria with higher salt tolerance can be incubated and grown. If 4% does not yield any colonies, it is possible to increase the concentration of salt in smaller increments, such as to 3.1% or 3.5%. When selecting tolerant colonies, it is important to test the bacteria for the continued presence and relative strength of antifungal activity, as such activity may be modified when grown under these conditions. Over many generations, it is possible to develop C60 bacteria that possess antifungal activity and are capable of growth under high osmotic pressure due to the presence of salt.

UV Tolerance.

UV tolerance is achieved by exposing C60 bacteria (spread onto agar plates using actively growing cultures) to very short doses of UV radiation, then incubating to see which colonies grow. This process is repeated over many generations to select for a tolerant mutant. A difficulty with this approach may be that it may be necessary to induce enzyme mechanisms capable of repairing UV-induced damage to obtain UV tolerant isolates. Also, exposure to UV could trigger spore production and as such would not induce UV tolerant mutants but rather induction of bacteria that are rapid spore formers.

An alternative approach is to introduce the genetic sequences used by UV tolerant bacteria to manufacture UV tolerance enzymes, into C60 by insertion

Endospore Production.

It would be possible to select for those C60 bacteria that are more rapidly able to produce an endospore in order to protect themselves from environmental damage. For example, C60 bacteria could be grown in favourable conditions until the cells are actively growing, then rapidly altering those conditions to unfavourable (such as by the introduction of heat, antibiotics, UV, salts etc.). Remaining vegetative cells are sterilised, (such as through pasteurization or UV sterilisation) and samples are inoculated onto fresh media. By collecting those colonies that are able to grow and repeating the process it would be possible to select those C60 bacteria that are more rapidly able to respond to unfavourable environmental changes by producing spores.

Antibiotic Resistance.

Antibiotic resistance could be induced by exposure of the C60 bacteria to sub-lethal concentrations of antibiotics for several generations of bacteria, followed by exposure of the surviving C60 bacteria to increasing levels of a given antibiotic.

Another possible method of development of antibiotic resistance is to grow the C60 bacteria alongside a population of resistant bacteria with the resistance plasmids, to permit plasmid transfer to occur, leading to antibiotic resistance.

Finally, because resistance is often plasmid mediated, it is possible that such resistance could be genetically engineered by transforming the C60 bacteria through the insertion of a plasmid carrying resistance.

Bacteria strain C60 was deposited with the Australian Government Analytical Laboratories at PO Box 385 Pymble, NSW 2073, Australia on 7 Jul. 2003 and given Accession no. NM03/36700.

It will be understood that the invention disclosed and defined herein extends to all alternative combinations of two or more of the individual features mentioned or evident from the text or drawings. All of these different combinations constitute various alternative aspects of the invention.

The foregoing describes embodiments of the present invention and modifications, obvious to those skilled in the art can be made thereto, without departing from the scope of the present invention.

1. (canceled)

2. An isolated Bacillus strain including a genome having at least 70% DNA-DNA similarity to the genome of a Bacillus strain according to Accession no. NM03/36700 and a melting temperature that is about the same as the melting temperature of a genome of a Bacillus strain according to Accession no. NM03/36700.

3. An isolated Bacillus strain according to Accession no. NM03/36700.

4. (canceled)

5. A bacteria for suppressing the growth of a fungus produced by the following steps:
(a) exposing a strain according to claim 2 to conditions to produce one or more mutant strains; and
(b) selecting a mutant strain that is capable of suppressing the growth of a fungus and that has a genome that has at least 70% DNA-DNA similarity to the genome of a Bacillus strain according to Accession no. NM03/36700 and a melting temperature that is about the same as the melting temperature of a genome of a Bacillus strain according to Accession no. NM03/36700.

6-9. (canceled)
